(19) United States
(12) Patent Application Publication
(10) Pub. No.: US 2025/0268733 A1
(43) Pub. Date: Aug. 28, 2025

(54) PROSTHETIC PYLON EMULATOR

(71) Applicant: Human Motion Technologies LLC, Pittsburgh, PA (US)

(72) Inventors: Joshua M. Caputo, Pittsburgh, PA (US); Carl S. Curran, Pittsburgh, PA (US); Tianyao Chen, Pittsburgh, PA (US)

(21) Appl. No.: 19/064,150

(22) Filed: Feb. 26, 2025

Related U.S. Application Data
(60) Provisional application No. 63/557,715, filed on Feb. 26, 2024.

(51) Int. Cl. A61F 2/78

(52) U.S. Cl. CPC A61F 2/7812; A61F 2002/7825

(57) ABSTRACT

A prosthetic pylon emulator for use with a user and an off-board actuation and control system, the user having a pylon socket, and the pylon emulator having a first frame, a second frame, a load cell, a driver, and a Bowden cable is disclosed. The first frame is operably attachable to a prosthesis, the second frame is operably attachable to the pylon socket of the user, and the second frame is slidable relative to the first frame. The first frame is operably coupled to an upper portion of the load cell and the second frame is operably coupled to a lower portion of the load cell. The driver is operably engaged with the first frame and the second frame, and the driver is configured to move the second frame from a retracted position to an extended position relative to the first frame upon actuation of the driver.

### PRIORITY CLAIM

This non-provisional application claims the benefit under 35 U.S.C. § 119 (e) of U.S. Provisional Patent Application Ser. No. 63/557,715, entitled PROSTHETIC PYLON EMULATOR, filed Feb. 26, 2024, the disclosure of which is incorporated by reference herein in its entirety.

### BACKGROUND

Components of a prosthetic limb include a pylon, which serves as the primary structural element connecting the socket, where the residual limb is secured, to the terminal device, such as a prosthetic foot or other functional appendages. The pylon transmits forces, providing stability, and enabling movement, all while supporting the user's body weight. The pylon's role extends beyond simple structural support; it often incorporates mechanisms to absorb shocks and torsional forces, simulating the biomechanical properties of a natural limb. These features reduce stress on the user's residual limb and improving overall comfort and mobility.

### SUMMARY

In one general aspect, the present invention is directed to a prosthetic pylon emulator for use with a user and an off-board actuation and control system, the user having a pylon socket, the prosthetic pylon emulator comprising a first frame, a second frame, a load cell, a driver, and a Bowden cable. The first frame is operably attachable to a prosthesis, the first frame comprising a base plate and a pair of upstanding sidewalls. The second frame is operably attachable to the pylon socket of the user, the second frame is slidable relative to the first frame. The first frame is operably coupled to an upper portion of the load cell and the second frame is operably coupled to a lower portion of the load cell. The driver is operably engaged with the first frame and the second frame, the driver is configured to move the second frame from a retracted position to an extended position relative to the first frame upon actuation of the driver. The second frame is positioned farther away from the first frame when the second frame is in the extended position than when the second frame is in the retracted position. The Bowden cable is operably engaged with the driver and the off-board actuation and control system. The off-board actuation and control system is configured to pull the Bowden cable in a first direction to actuate the driver and move the second frame from the retracted position to the extended position.

In various aspects, the pylon emulators disclosed herein are designed to provide one degree-of-freedom (DoF) linear motion along the length of an amputee's leg, similar to a standard prosthetic pylon with vertical compression capability, resisting bending and twisting loads in any other direction. The pylon emulators disclosed herein can be configured to operate with commercial prosthetic feet or with other prosthesis emulators. The pylon emulators disclosed herein have the ability to exhibit software-controllable force-deflection characteristics, with a range and precision of adjustability far beyond conventional prosthetic pylon technology.

In at least one aspect, the pylon emulators disclosed herein are designed with a single axial DOF along the pylon of a transtibial amputee to control the instantaneous longitudinal stiffness, force, and/or displacement. The pylon emulators disclose herein may have programmable force-deflection behavior (linear/non-linear spring, time based force profile, etc.) using onboard sensors (load sensor/strain gauge/load cell with position sensor/string potentiometer) and off board actuation and control. These and other benefits that can be realized through various embodiments of the present invention will be apparent from the description that follows.

### BRIEF DESCRIPTION OF THE DRAWINGS

Various features of the embodiments described herein, together with advantages thereof, may be understood in accordance with the following description taken in conjunction with the accompanying drawings as follows:

FIG. 1 is an exploded view of a prosthetic pylon emulator and prostheses for use with the prosthetic pylon emulator, in accordance with at least one aspect of the present invention.

FIG. 2 is a front view of a driver of the prosthetic pylon emulator of FIG. 1, in accordance with at least one aspect of the present invention.

FIG. 3 is an exploded view of the prosthetic pylon emulator of FIG. 1, in accordance with at least one aspect of the present invention.

FIG. 4 is a side view of the prosthetic pylon emulator of FIG. 1 being used with an off-board actuation and control system including a display, in accordance with at least one aspect of the present invention.

FIG. 5 is a side cross section view of the prosthetic pylon emulator of FIG. 1, illustrating a superior frame of the prosthetic pylon emulator in a retracted position, in accordance with at least one aspect of the present invention.

FIG. 6 is a side cross section view of the prosthetic pylon emulator of FIG. 1, illustrating the superior frame of the prosthetic pylon emulator in an extended position, in accordance with at least one aspect of the present invention.

FIG. 7 is a front view of the prosthetic pylon emulator of FIG. 1, illustrating the superior frame of the prosthetic pylon emulator in the retracted position, in accordance with at least one aspect of the present invention.

FIG. 8 is a front view of the prosthetic pylon emulator of FIG. 1, illustrating the superior frame of the prosthetic pylon emulator in the extended position, in accordance with at least one aspect of the present invention.

FIG. 9 is a perspective view of the prosthetic pylon emulator of FIG. 1, illustrating a string potentiometer operably attached to the prosthetic pylon emulator, in accordance with at least one aspect of the present invention.

FIG. 10 is a front view of the prosthetic pylon emulator of FIG. 1, illustrating the string potentiometer, in accordance with at least one aspect of the present invention.

FIG. 11 is a perspective view of the prosthetic pylon emulator of FIG. 1, illustrating a hook extending from the superior frame and bolts extending from a inferior frame, in accordance with at least one aspect of the present invention.

FIG. 12 is a perspective view of the prosthetic pylon emulator of FIG. 1, illustrating an elastic band operably attached to the hook and bolts, in accordance with at least one aspect of the present invention.

FIG. 13 is a perspective view of the prosthetic pylon emulator of FIG. 1, illustrating a compression spring arrangement between the superior frame and the inferior frame, in accordance with at least one aspect of the present invention.

FIG. 13A is a perspective view of the prosthetic pylon emulator of FIG. 1, illustrating a leaf spring arrangement between the superior frame and the inferior frame with the superior frame in the retracted position, in accordance with at least one aspect of the present invention.

FIG. 13B is a perspective view of the prosthetic pylon emulator of FIG. 1, illustrating a leaf spring arrangement between the superior frame and the inferior frame with the pylon emulator in the extended position, in accordance with at least one aspect of the present invention.

FIG. 14 is a perspective view of another prosthetic pylon emulator, in accordance with at least one aspect of the present invention.

FIG. 15 is a perspective view of the prosthetic pylon emulator of FIG. 14, illustrating a driver of the emulator as a ball screw assembly(ies), in accordance with at least one aspect of the present invention.

FIG. 16 is a cross section view of the prosthetic pylon emulator of FIG. 14, illustrating the superior frame in a retracted position, in accordance with at least one aspect of the present invention.

FIG. 17 is a cross section view of the prosthetic pylon emulator of FIG. 14, illustrating the superior frame in an extended position, in accordance with at least one aspect of the present invention.

FIG. 18 is a cross section plan view of the prosthetic pylon emulator of FIG. 14, illustrating a gear arrangement and a ball screw(s) of the ball screw assembly(ies), in accordance with at least one aspect of the present invention.

FIG. 19 is a cross-section view of the prosthetic pylon emulator of FIG. 14, illustrating a spindle operably engaged with the gear arrangement, in accordance with at least one aspect of the present invention.

FIG. 20 is a plan view of another ball screw assembly, illustrating the ball screw assembly operably engaged with the gear arrangement of the prosthetic pylon emulator of FIG. 14, in accordance with at least one aspect of the present invention.

FIG. 21 is a cross section side view of FIG. 20, in accordance with at least one aspect of the present invention.

FIG. 22 is a cross section side view of the ball screw assembly of FIG. 20 directly engaged with a drive screw of the drive screw(s) of the prosthetic pylon emulator of FIG. 14, in accordance with at least one aspect of the present invention.

FIG. 23 is a cross section front view of a central ball screw assembly, illustrating the central ball screw assembly operably engaged with a superior and inferior frame portion of a pylon emulator, in accordance with at least one aspect of the present invention.

FIG. 24 is a cross section front view of the prosthetic pylon emulator of FIG. 14, illustrating a biasing member attached to the superior frame portion and the inferior frame portion, in accordance with at least one aspect of the present invention.

FIG. 25 is a perspective view of a load cell positioned intermediate an inferior and superior frame portion of a prosthetic pylon emulator, in accordance with at least one aspect of the present invention.

FIG. 26 is a graph of pylon displacement versus amplification ration that was generated during testing of the prosthetic pylon emulator of FIG. 1, in accordance with at least one aspect of the present invention.

FIG. 27 is a perspective view of a testing rig for use with a prosthetic pylon emulator such as the prosthetic pylon emulator of FIG. 1, in accordance with at least one aspect of the present invention.

FIG. 28 is a graph depicting a pylon stiffness emulation generated during testing of the prosthetic pylon emulator of FIG. 1, in accordance with at least one aspect of the present invention.

### DETAILED DESCRIPTION

Pylon prostheses involve the use of a structure, oftentimes tubular in geometry, positioned between a socket and a foot to provide a weight-bearing, shock-absorbing support shaft for a prosthetic device. Connecting parts of a prosthesis is critical to building a stable, reliable prosthesis for a particular user. Pylons must be able to withstand a user's weight and/or the weight of routinely-carried loads. Shock-absorbing pylons are components that increase prosthetic compliance and/or provide shock absorption during walking, running, and/or during other high-impact activities. Shock-absorbing pylons are designed to attenuate impact forces by compressing, for example, to absorb, return, and/or dissipate energy. While current shock-absorbing pylons can attenuate residual leg impact, such pylons fail to optimize an interaction with ankle and/or foot components.

Given the unique nature surrounding each particular user of a prosthetic device, it is desirable to design, test, and/or fine-tune attributes of the various components of the prosthesis prior to prescribing a final prosthesis to each user. Emulator-based approaches serve to improve individualization of prosthesis prescription and/or accelerate the development of novel prosthetic and orthotic devices, for example. The prosthetic pylon emulators disclosed herein allow for rapid testing of mechanical properties such as stiffness and power, for example, to facilitate prescription to maximize locomotion performance of a particular user.

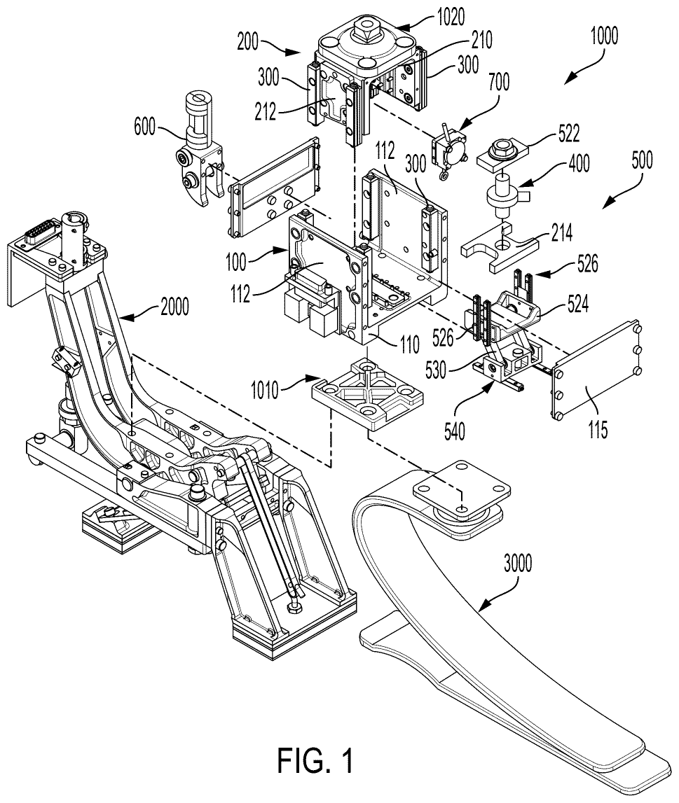

FIGS. 1-13 depict a tethered, wearable prosthetic pylon testbed, or prosthetic pylon emulator 1000 according to various embodiments of the present invention. Referring to FIG. 1, the pylon emulator 1000 includes a first frame 100 (e.g., an inferior frame) operably attachable to a prosthesis, such as the prosthesis 2000 or the prosthesis 3000. In various aspects, an adapter 1010 may be used to removably attach the prosthesis 2000, 3000 to the first frame 100. In various aspects, the pylon emulator 1000 is modular so as to be compatible, or otherwise operable, with commercial prosthetic feet and/or with prosthesis emulators, for example. In at least one aspect, the pylon emulator 1000 is fitted with the interchangeable prosthetic adapter 1010 on a surface of the pylon emulator 1000 intended to be positioned adjacent a modular foot component. The interchangeable prosthetic adapter 1010 allows for the pylon emulator 1000 to be secured, or otherwise coupled to, any desired commercial prosthetic foot and/or robotic prosthetic emulator, for example. Stated another way, the prosthetic adapter 1010 can be swapped out to include the appropriate fittings and/or couplings for the desired foot component. In addition, or alternatively, to the prosthetic adapter 1010, the pylon emulator 1000 can include an industry-standard pyramid adapter 1020 to facilitate coupling of the pylon emulator to a desired foot component.

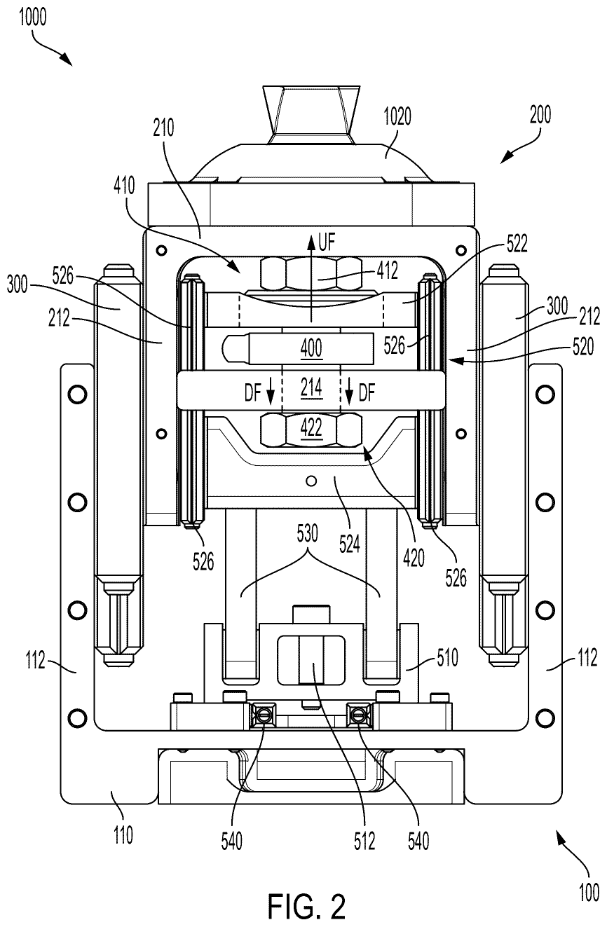

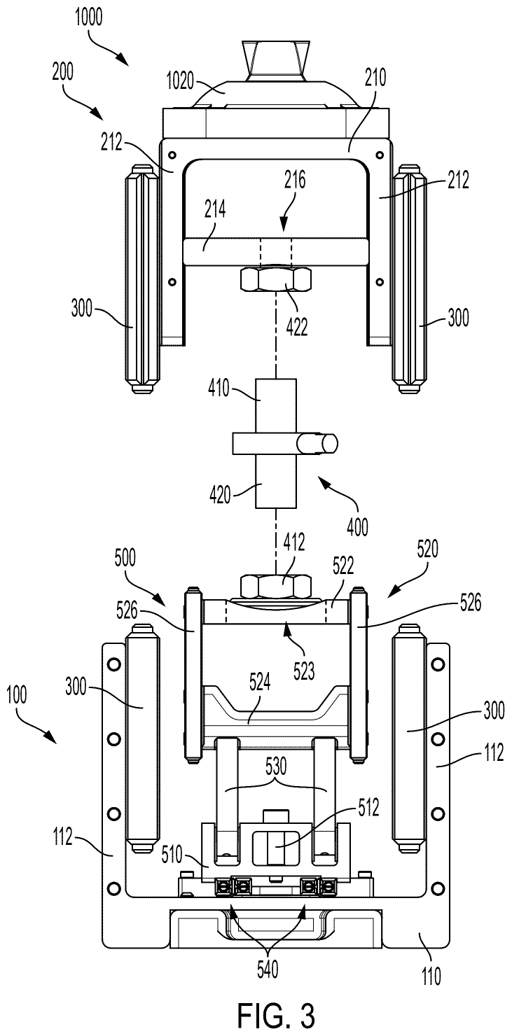

Referring to the exemplary embodiment shown in FIGS. 2 and 3, the first frame 100 includes a base plate 110 and a pair of upstanding sidewalls 112. The pylon emulator 1000 includes a second frame 200 (e.g., a superior frame) operably attachable to a pylon socket (not shown) of the user. In at least one aspect, the pyramid adapter 1020 is attached to the second frame 200 and the pyramid adapter 1020 is attachable to the pylon socket of the user. Further, the second frame 200 fits within and is slidable relative to the first frame 100. The second frame 200 includes a top plate 210, a pair of downwardly extending sidewalls 212, and a retention plate 214. The pylon emulator 1000 further comprises first bearing slides 300 positioned intermediate the first frame 100 and the second frame 200 to permit the second frame 200 to slide relative to the first frame 100. Preferably, one half of each of the bearing slides 300 is attached to the first frame 100 and the other half of each of the bearing slides 300 are attached to the second frame 200, the two halves of the bearing slides 300 are engaged with each other to permit the second frame 200 to slide relative to the first frame 100. In the illustrated embodiment, four bearing slides 300 are shown, however any suitable number of bearing slides 300 may be employed.

Further to the above, the pylon emulator 1000 may include a load cell 400 operably coupled with the first frame 100 and operably coupled with the second frame 200. A load cell is a type of sensor or transducer that converts force, typically a weight, into an electrical signal. This signal can then be measured, displayed, and used for control or monitoring purposes. Suitable load cell types include strain gauge load cells, hydraulic load cells, pneumatic load cells, and piezoelectric load cells, for example. In at least one aspect, the first frame 100 is operably coupled to an upper portion 410 (see FIGS. 2 and 3) of the load cell 400 and the second frame 200 is operably coupled to a lower portion 420 (see FIGS. 2 and 3) of the load cell 400.

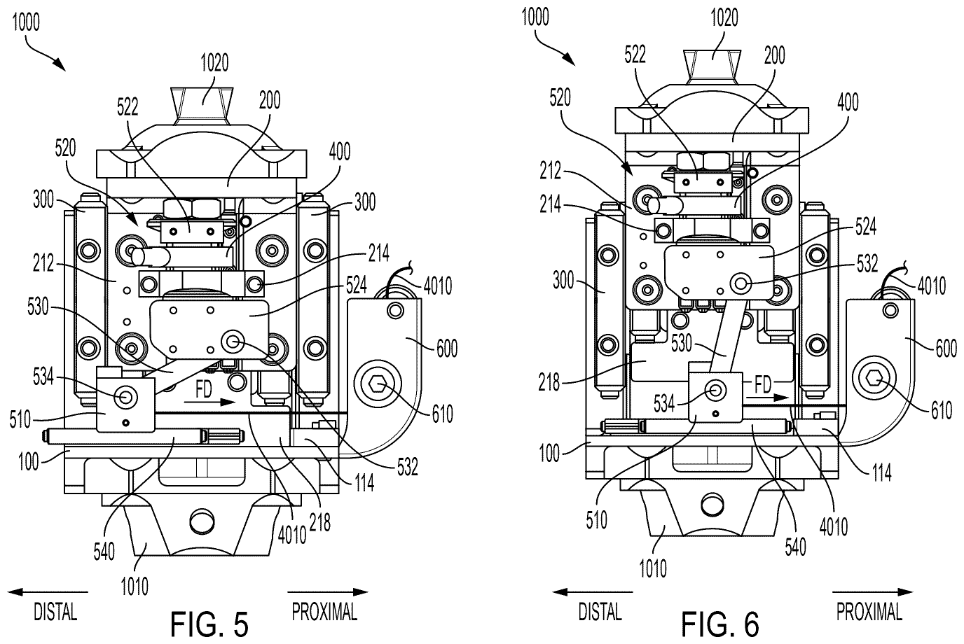

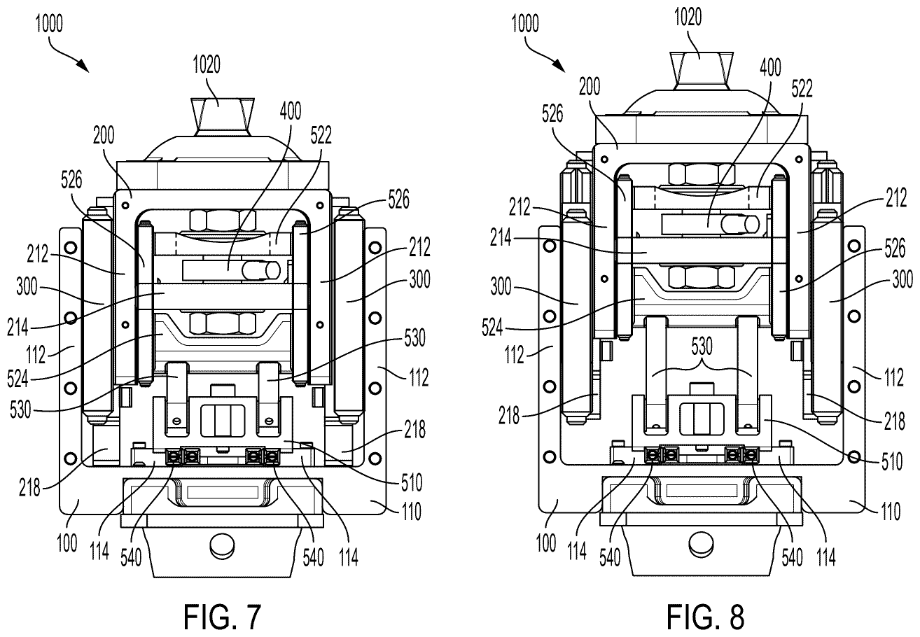

Further to the above, the pylon emulator 1000 can further comprise a driver (e.g., an actuation mechanism) operably engaged with the first frame 100 and the second frame 200. The driver is configured to move the second frame 200 from a retracted position (FIGS. 5 and 7) to an extended position (FIGS. 6 and 8) relative to the first frame 100 upon actuation of the driver. In at least one aspect, the second frame 200 is positioned farther away from the first frame 100 when the second frame 200 is in the extended position than when the second frame 200 is in the retracted position. Referring to FIG. 1, in at least one aspect, the driver may be a carriage assembly 500. However, alternative embodiments for moving the second frame 200 from the retracted position to the extended position relative to the first frame 100 are described herein.

Referring again to the example shown in FIGS. 2 and 3, the carriage assembly 500 includes a base carriage 510, an upper carriage 520 slidably attached to the second frame 200, and an arm(s) 530 (e.g., one or more than one arm) rotatably attached to the base carriage 510 and rotatably attached to the upper carriage 520. The base carriage 510 is linearly translatable relative to the base plate 110 of the first frame 100 between a distal position (FIG. 5) and a proximal position (FIG. 6). In at least one aspect, the base carriage 510 is slidably attached to the base plate 110 of the first frame 100 by way of one or more than one linear slide 540.

Further to the above, the upper carriage 520 can include an upper plate 522, a lower plate 524, and a plurality of second bearing slides 526 connecting the upper plate 522 to the lower plate 524, the plurality of second bearing slides 526 are positioned intermediate the upper carriage 520 and the downwardly extending sidewalls 212 of the second frame 200. The second bearing slides 526 are positioned on either lateral side of the upper carriage 520 as shown in FIGS. 2 and 3. In various aspects, the second bearing slides 526 permit the upper carriage 520 to linearly translate relative to the second frame 200.

Referring to the example shown in FIGS. 5 and 6, the arm(s) 530 is rotatably attached to the lower plate 524 of the upper carriage 520 by way of a pin 532 and the arm(s) 530 is rotatable attached to the base carriage 510 by way of a pin 534. In use, as the base carriage 510 translates from the distal position (FIG. 5) to the proximal position (FIG. 6), the arm(s) 530 lifts the upper carriage 520 relative to the base carriage 510 and moves the second frame 200 from the retracted position (FIG. 5) to the extended position (FIG. 6).

Referring to the example shown in FIGS. 2 and 3, the retention plate 214 of the second frame 200 is positioned intermediate the upper plate 522 and the lower plate 524 of the upper carriage 520. The upper portion 410 of the load cell 400 extends through a hole 523 defined through the upper plate 522 of the upper carriage 520, and the lower portion 420 of the load cell 400 extends through a hole 216 defined through the retention plate 214. The through holes 216, 523 are shown as hidden lines in FIGS. 2 and 3. Further, the upper portion 410 of the load cell 400 is attached to a nut 412 positioned above the upper plate 522 and the lower portion 420 of the load cell 400 is attached to a nut 422 positioned below the retention plate 214. As such, the upper portion 410 of the load cell 400 is retained to the upper plate 522 and the lower portion 420 of the load cell 400 is retained to the retention plate 214. Owing to this arrangement, the load cell 400 is capable of being placed in tension and is configured to measure the force applied by the upper carriage 520 to the second frame 200, as discussed in greater detail below.

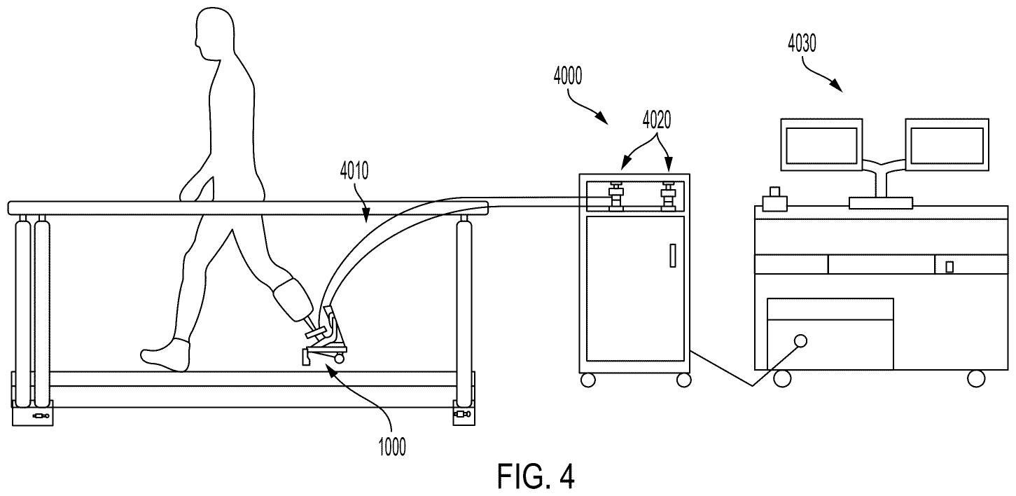

Referring to the example shown in FIGS. 5 and 6, a rope or wire 4010 (e.g., a Bowden cable) may be attached to the base carriage 510 by way of a pin 512 (see FIG. 2). A Bowden cable is a type of flexible mechanical cable used to transmit force or motion between two points. It typically consists of an inner wire or cable that slides within an outer sheath or housing. In at least one aspect, the cable 4010 is fixedly attached to the base carriage 510. In at least one aspect, the cable 4010 is wrapped around the pin 512 of the base carriage 510 to attach the cable 4010 to the base carriage 510. The Bowden cable 4010 is configured to be pulled in a first direction FD using an off board actuation and control system 4000 (see FIG. 4) to translate the base carriage 510 from the distal position (FIG. 5) to the proximal position (FIG. 6). In at least one aspect, the base carriage 510 is moved from the distal position to the proximal position in response to the off-board actuation and control system 4000 pulling the Bowden cable in the first direction FD. U.S. Pat. No. 12,127,995, assigned to Human Motion Technologies LLC and which is incorporated herein by reference in its entirety, provides details regarding an exemplary off-board actuation and control system. Further, the arm(s) 530 of the carriage assembly 500 is configured to transition from a collapsed configuration (FIGS. 5 and 7) to an erect configuration (FIGS. 6 and 8) in response to the base carriage 510 moving from the distal position (FIG. 5) to the proximal position (FIG. 6). Further, the upper carriage 520 moves away from the base carriage 510 (e.g., is raised) to move the second frame 200 from the retracted position in FIG. 5 to the extended position in FIG. 6 in response to the arm(s) 530 transitioning from the collapsed configuration to the erect configuration. As such, the second frame 200 moves from the retracted position (FIGS. 5 and 7) to the extended position (FIGS. 6 and 8) in response to the Bowden cable 4010 being pulled a first amount in the first direction FD. In various aspects, the carriage assembly 500 redirects the pulling force from the cable tether, or Bowden cable, to a pushing force that resists the weight of the user.

As discussed above, the upper carriage 520 preferably is slidably attached to the second frame 200 by way of the second bearing slides 526. Further, the upper plate 522 and the lower plate 524 of the upper carriage 520 are connected to each other by way of the bearing slides 526. As such, the upper carriage 520 and, thus, the upper plate 522 and the lower plate 524 are translatable relative to the retention plate 214 of the second frame 200 which permits the load cell 400 to be placed in tension when the Bowden cable 4010 is pulled in the first direction FD. Specifically, as the Bowden cable 4010 is pulled in the first direction FD, the upper plate 522 will move away from the retention plate 214. When the upper plate 522 moves away from the retention plate 214, the upper plate 522 will apply an upward force UF (FIG. 2) to the upper portion 410 of the load cell 400 and the retention plate 214 will apply a downward force DF (FIG. 2) to the lower portion 420 of the load cell 400. In such instances, the load cell 400 is placed in tension. The load cell 400 is configured to measure the amount of force applied by the upper carriage 520 to the second frame 200 in response to the off board actuation and control system 4000 pulling the Bowden cable 4010 the first amount in the first direction FD. In various aspects, the load cell 400 maintains constant tension with the carriage assembly 500 with no extraneous loads. The load cell 400 serves to directly measure the linear force from the compression of the emulator 1000 as a load, or weight, is applied to the second frame 200 of the pylon emulator 1000. In at least one aspect, only the force applied by the upper carriage 520 to the second frame 200 is measured by the load cell 400. Further, in at least one aspect, the load cell 400 is configured to experience a purely tensile force owing to the arrangement of the carriage assembly 500 relative to the load cell 400. Specifically, the carriage assembly 500 is split between the base carriage 510 and the upper carriage 520, the upper carriage 520 being movable relative to the second frame 200.

In various aspects, the pylon emulators described herein are powered and controlled by an off-board actuation and control system which may be any one of the Humotech Full-Sized Caplex System, the Humotech Portable Caplex System, and/or the Humotech Open Source Leg System. FIG. 4 illustrates one example of an off board actuation and control system 4000. As shown in FIG. 4, the pylon emulator 1000 is tethered and/or otherwise coupled to off-board motors 4020 by one or more cables 4010 used to transmit mechanical force or energy, such as Bowden cables, for example. The pylon emulator 1000 is further coupled to a display 4030 by any suitable electrical connection.

Referring to FIGS. 5 and 6, in at least one aspect, the pylon emulator 1000 includes a housing 600 (e.g., a Bowden Cable termination) for routing the Bowden cable 4010 from the off-board actuation and control system 4000 to the base carriage 510. The housing 600 includes an idler shaft 610 that is configured to route the Bowden cable 4010 ninety degrees to connect with the base carriage 510. As discussed above, a Bowden cable typically consists of an inner wire or cable that slides within an outer sheath or housing. In at least one aspect, the Bowden cable 4010 comprises a housing or sheath that terminates at the housing 600, and the inner wire or cable extends through the housing 600 and attaches to the base carriage 510.

Further to the above, the pylon emulator 1000 can be personalized for each particular user to limit a range of motion within a desired zone. Such a unique range of motion can be customized and modeled using hard stops and/or bumpers, for example, as described in greater detail below.

In at least one aspect, the second frame 200 includes one or more than one range of motion (ROM) limiting bumper 218 attached to at least one of the downwardly extending sidewalls 212 of the second frame 200, as shown in FIGS. 7 and 8. In at least one aspect, there are at least two bumpers 218, where one of the bumpers 218 extends downward from each of the sidewalls 212. In use, the bumpers 218 are configured to abut the base plate 110 of the first frame 100 when the second frame 200 is in the retracted position (FIG. 7). The position of the bumpers 218 can be adjusted relative to the sidewalls 212. For example, the bumpers 218 may be bolted to the sidewalls 212 in various axial locations along the direction of travel of the second frame 200. The position of the bumpers 218 relative to the second frame 200 sets the retracted position of the second frame 200 relative to the first frame 100.

Further to the above, in at least one aspect, the first frame 100 includes one or more than one hard stop 114 attached to the base plate 110 of the first frame 100 (see FIGS. 5 and 6). In use, the base carriage 510 is configured to abut the hard stops 114 when the base carriage 510 is in the proximal position (FIG. 6). The hard stops 114 prevent the base carriage 510 from moving further in the proximal direction beyond the hard stops 114. As such, the position of the hard stops 114 relative to the first frame 100 and the base carriage 510 determines the maximum amount the second frame 200 can be extended relative to the first frame 100. In other words, the hard stops 114 set a maximum extended position of the second frame 200. In various aspects, the position of the hard stops 114 relative to the first frame 100 can be adjusted to adjust the maximum amount the second frame 200 can be extended relative to the first frame 100. In various aspects, the adjustable hard stops 114 serve to limit the extended position of the second frame 200 relative to the first frame 100 while the adjustable bumpers 218 serve to limit the retracted position of the second frame 200 relative to the first frame 100. In other words, the hard stops 114 and the bumpers 218 limit the distance (e.g., the stroke distance) that the second frame 200 travels from the retracted position to the extended position). Any suitable number and/or combination of hard stops and/or bumpers can be utilized in the pylon emulator 1000.

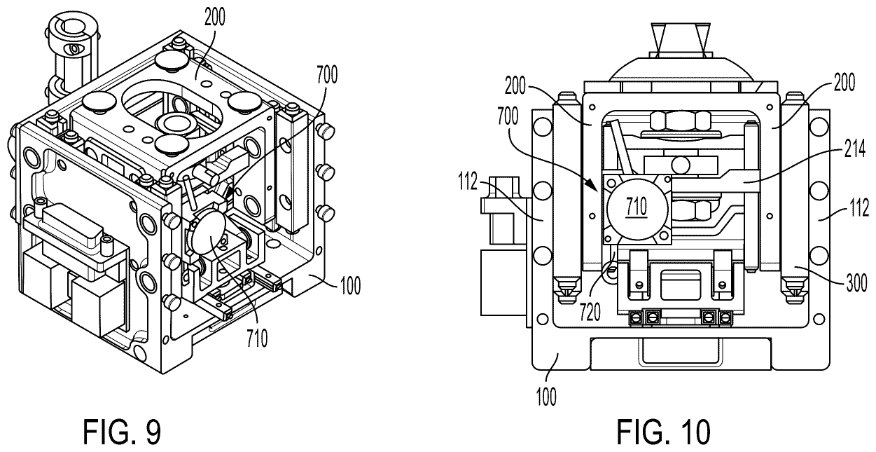

Referring to the example of FIGS. 9 and 10, the pylon emulator 1000 includes a string potentiometer 700 to actively measure the extension and compression of the pylon emulator 1000 (e.g., actively measure the travel distance and velocity of the second frame 200 relative to the first frame 100). In at least one aspect, a body 710 of the string potentiometer 700 is attached either to the first frame 100 or the second frame 200 and a string, or wire 720, is attached to the other one of the first frame 100 or the second frame 200. In the illustrated embodiment, the body 710 is attached to the retention pate 214 of the second frame 200 and the wire 720 is attached to a front plate 115 (see FIG. 1) of the first frame 100. The front plate 115 is attached to the pair of upstanding sidewalls 112 of the first frame 100, for example. In use, as the second frame 200 moves from the retracted position toward the extended position, the wire 720 will unreel creating an electrical signal in the body 710 that is proportional to the linear extension and velocity of the wire 720. The electrical signal can be read (e.g., measured) by the actuation and control system 4000. The electrical signal measure is proportional to the linear extension and velocity of the second frame 200 as the second frame 200 slides relative to the first frame 100. In other words, the amount of linear extension of the cable and/or the velocity of the cable is directly proportional to the distance the upper frame moves away from the lower frame and the velocity of the upper frame, respectively. In at least one aspect, the second frame 200 moves a stroke distance from the retracted position toward the extended position in response to the off board actuation and control system 4000 pulling the Bowden cable 4010 a first amount in the first direction FD. The string potentiometer 700 is configured to determine the stroke distance of the second frame 200 and/or the velocity of the second frame 200 based upon the off board actuation and control system 4000 pulling the Bowden cable 4010 the first amount.

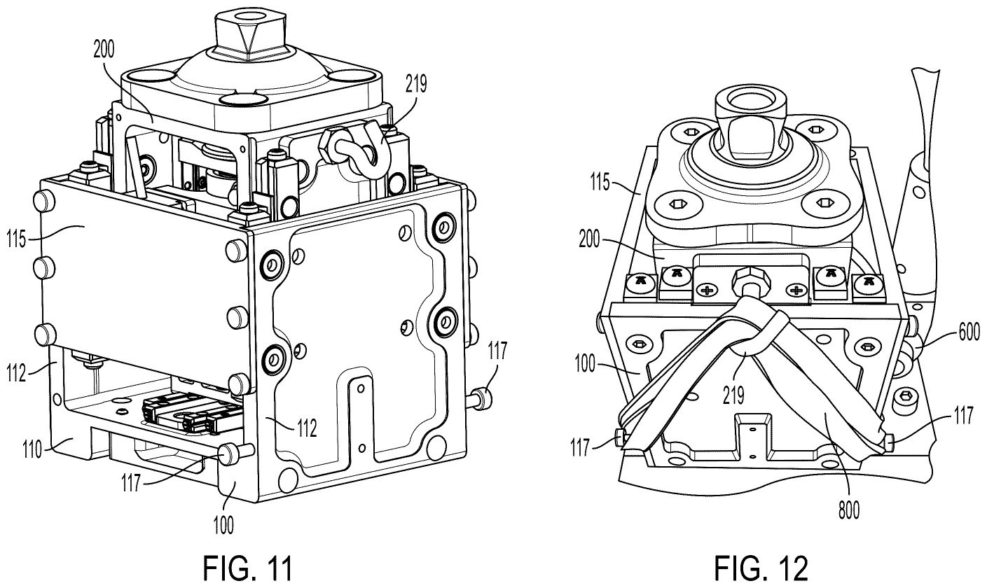

Referring to FIGS. 11 and 12, in at least one aspect, the second frame 200 includes a protrusion (e.g., a hook 219) extending from at least one of the sidewalls 212 of the second frame. Further, the first frame 100 includes a pair protrusions (e.g., bolts 117 extending from the sidewalls 112 of the first frame 100. Referring to FIG. 12, a biasing member 800 (e.g., an elastic band) can be seated around the hook 219 and looped around the bolts 117. As such, the biasing member 800 is removably attachable to the first frame 100 and/or is removably attachable to the second frame 200. The biasing member 800 biases the second frame 200 toward the retracted position. In other words, the biasing member 800 biases the second frame 200 toward the base plate 110 of the first frame 100. In use, when the Bowden cable 4010 is being pulled in the first direction FD to move the second frame 200 from the retracted position toward the extended position, the force applied by the biasing member 800 to the second frame 200 is overcome and the second frame 200 moves toward the extended position. When the Bowden cable 4010 is not being actively pulled in the first direction FD and the second frame 200 is in the extended position, the biasing force of the biasing member 800, along with the weight of the user, will move the second frame 200 from the extended position to the retracted position. As such, in various aspects, the biasing member 800 aids the user in returning the second frame 200 to the retracted position. Specifically, in instances where a user's extremity is operably attached to the second frame 200, the biasing member 800 can aid the user in returning the second frame 200 to the retracted position. The biasing member 800 arrangement described herein may be used with any of the pylon emulators described herein.

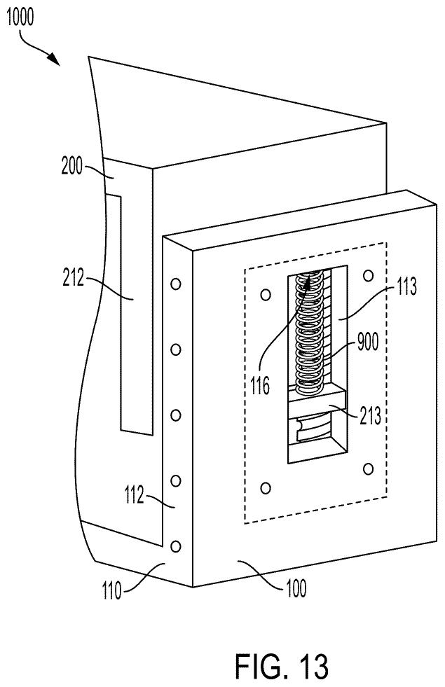

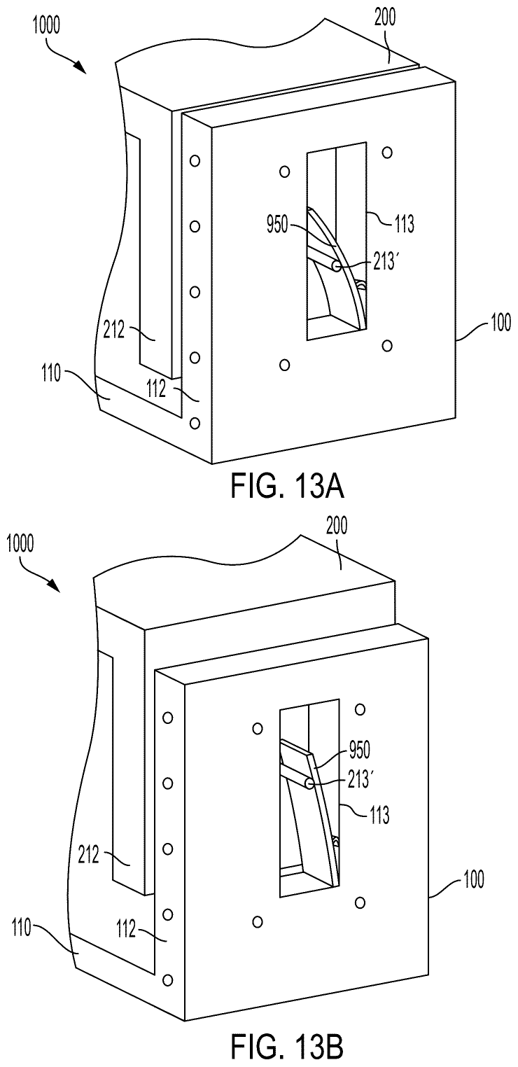

Referring to FIG. 13, in at least one aspect, the pylon emulator 1000 may include a compression spring 900 positioned intermediate the first frame 100 and the second frame 200 as an alternative, or in addition to, the biasing member 800. In at least one aspect, the second frame 200 includes an extension tab 213 extending from one of the downwardly extending sidewalls 212. The extension tab 213 extends into an opening 113 defined in one of the upstanding sidewalls 112 of the first frame 100 that is adjacent the sidewall 112 of the second frame 200. The compression spring 900 is positioned intermediate the extension tab 213 and an upper surface 116 of the opening 113. In use, the compression spring 900 biases the second frame 200 toward the retracted position. In other words, the compression spring 900 biases the second frame 200 toward the base plate 110 of the first frame 100. In at least one aspect, the same arrangement can be provided on the other side of the pylon emulator 1000 such that two compression springs are utilized to bias the second frame 200 toward the retracted position. In use, when the Bowden cable 4010 is being pulled in the first direction FD to move the second frame 200 from the retracted position toward the extended position, the force applied by the compression spring 900 to the second frame 200 is overcome and the second frame 200 moves toward the extended position. When the Bowden cable 4010 is not being actively pulled in the first direction FD and the second frame 200 is in the extended position, the biasing force of the compression spring 900, along with the weight of the user, will move the second frame 200 from the extended position to the retracted position. As such, in various aspects, the compression spring aids the user in returning the second frame 200 to the retracted position. Specifically, in instances where a user's extremity is operably attached to the second frame 200, the compression spring 900 can aid the user in returning the second frame 200 to the retracted position. The compression spring 900 arrangement described herein may be used with any of the pylon emulators described herein.

Referring to FIGS. 13A and 13B, in at least one aspect, the pylon emulator 1000 may include a leaf spring 950 attached to the first frame 100 and extending into the opening 113 defined in the first frame 100. The leaf spring 950 may be an alternative, or in addition to, the biasing member 800 and/or the compression spring 900. In at least one aspect, the second frame 200 includes a protrusion 213′ (e.g., a pin) extending from one of the downwardly extending sidewalls 212. The protrusion 213′ extends into the opening 113 defined in the upstanding sidewall 112 of the first frame 100 that is adjacent the sidewall 212 of the second frame 200. The leaf spring 950 is positioned adjacent to, and in contact with, the protrusion 213′ of the second frame 200 when the pylon emulator 1000 is in the retracted configuration (FIG. 13A). In use, the leaf spring 950 biases the second frame 200 toward the retracted position. In other words, the leaf spring 950 biases the second frame 200 toward the base plate 110 of the first frame 100. In at least one aspect, the same arrangement can be provided on the other side of the pylon emulator 1000 such that two leaf spring 950 and two protrusions 213′ are utilized to bias the second frame 200 toward the retracted position. In use, when the Bowden cable 4010 is being pulled in the first direction FD to move the second frame 200 from the retracted position (FIG. 13A) toward the extended position (FIG. 13B), the force applied by the leaf spring 950 to the second frame 200 is overcome and the second frame 200 moves toward the extended position (FIG. 13B). When the Bowden cable 4010 is not being actively pulled in the first direction FD and the second frame 200 is in the extended position, the biasing force of the leaf spring 950, along with the weight of the user, will move the second frame 200 from the extended position to the retracted position. As such, in various aspects, the leaf spring 950 aids the user in returning the second frame 200 to the retracted position. Specifically, in instances where a user's extremity is operably attached to the second frame 200, the leaf spring 950 can aid the user in returning the second frame 200 to the retracted position. The leaf spring 950 arrangement described herein may be used with any of the pylon emulators described herein.

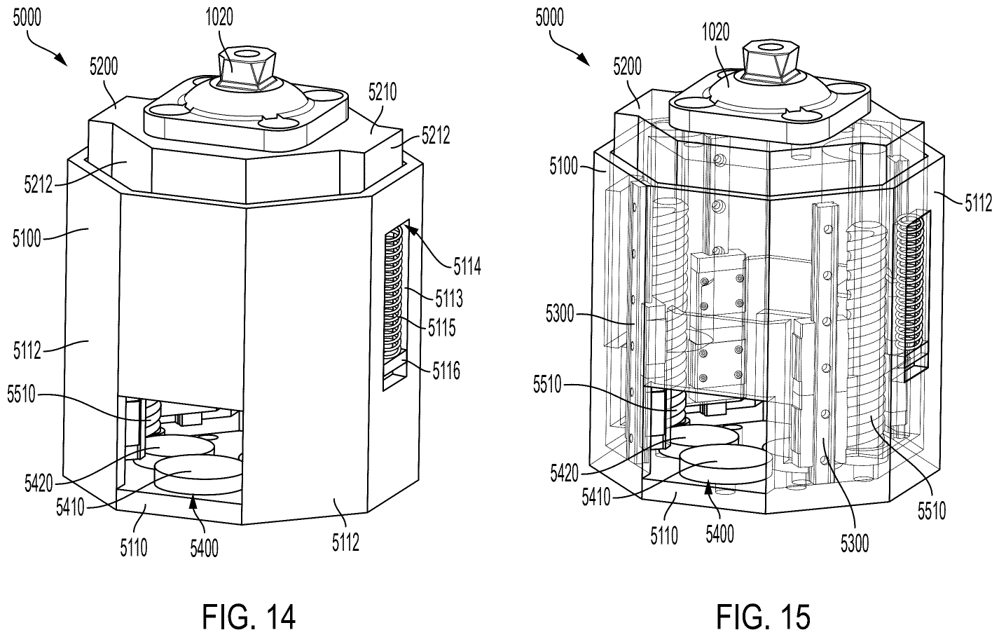

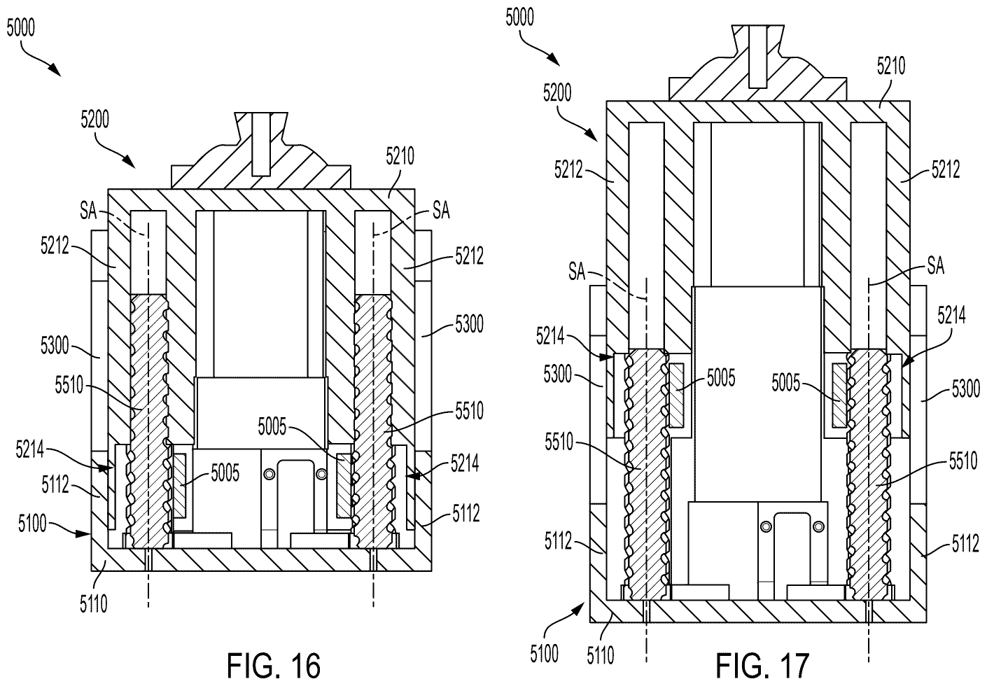

FIGS. 14-24 illustrate a prosthetic pylon emulator 5000 according to various embodiments of the present invention. The pylon emulator 5000 is similar to the pylon emulator 1000, although having the differences discussed herein. In at least one aspect, the pylon emulator 5000 utilizes a different driver (e.g., one or more than one ball screw assembly) to actuate a superior frame relative to an inferior frame in place of the driver (e.g., the carriage assembly 500) of the pylon emulator 1000 described above. Referring to FIGS. 14-17, the pylon emulator 5000 includes a first frame 5100 and a second frame 5200. The first frame 5100 includes a base plate 5110 and upstanding walls 5112 extending from the base plate 5110. The second frame 5200 includes a top plate 5210 and downwardly extending walls 5212 extending from the top plate 5210. The second frame 5200 is slidable relative to the first frame 5100 between a retracted position (FIG. 16) and an extended position (FIG. 17). In at least one aspect, a plurality of bearing slides 5300 are positioned intermediate the first frame 5100 and the second frame 5200 to permit the second frame 5200 to slide relative to the first frame 5100. The pylon emulator 5000 includes a driver (e.g., one or more than one ball screw assembly 5500). The one or more than one ball screw assembly 5500 is operably engaged with a Bowden cable, such as the Bowden cable 4010 described herein. The Bowden cable is configured to actuate the one or more than one ball screw assembly 5500 to move the second frame 5200 from the retracted position to the extended position, as discussed in greater detail below.

Each of the one or more than one ball screw assembly 5500 includes a drive screw 5510 rotatably attached to the first frame 5100 and operably engaged with the Bowden cable 4010. Specifically, the drive screw 5510 is rotatably attached to the base plate 5110 of the first frame 5100 such that the drive screw 5510 is rotatable about a screw axis SA defined by the drive screw 5510 relative to the first frame 5100. Further, the drive screw 5510 extends into an internal housing 5214 defined in the second frame 5200. The one or more than one ball screw assembly 5500 includes a plurality of ball bearings (not shown) positioned intermediate the drive screw 5510 and the internal housing 5214 of the second frame 5200. The ball bearings are configured to drive the second frame 5200 from the retracted position to the extended position in response to rotation of the drive screw 5510 owing to their engagement with the internal housing 5214 of the second frame 5200.

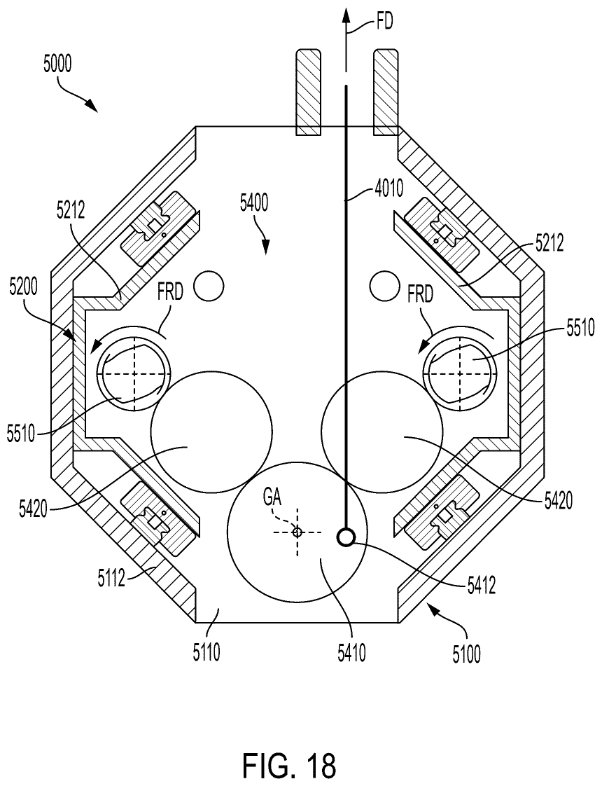

In use, the one or more than one drive screw 5510 is configured to rotate in a first rotary direction FRD (see FIG. 18) about its screw axis SA in response to an off-board actuation and control system (e.g., the actuation and control system 4000) pulling a Bowden cable (e.g., the Bowden cable 4010) in a first direction. Rotation of the drive screw in the first rotary direction FRD moves the second frame 5200 from the retracted position (FIG. 16) toward the extended position (FIG. 17). Several embodiments for effectuating rotation of the one or more than one drive screw 5510 are described in greater detail below.

In at least one aspect, the pylon emulator 5000 includes a gear arrangement 5400 positioned on the base plate 5110 of the first frame 5100. The gear arrangement 5400 includes a drive gear 5410 rotatably attached to the base plate 5110 of the first frame 5100. The drive gear 5410 is rotatable relative to the base plate about a gear axis GA (see FIG. 18). In at least one aspect, a Bowden cable (e.g., the Bowden cable 4010) is configured to rotate the drive gear in response to an off-board actuation and control system (e.g., the control system 4000) pulling the Bowden cable 4010 in the first direction FD. In at least one aspect, the Bowden cable 4010 is fixed to the drive gear 5410 at a point 5412 that is off center from the gear axis GA, as shown in FIG. 18. Further, the gear arrangement 5400 includes one or more than one intermediate gear 5420 rotatably attached to the base plate 5110 of the first frame 5100. Each intermediate gear 5420 is engaged with the drive gear 5410 and a respective one of the drive screws 5510. The intermediate gears 5420 are configured to rotate their respective drive screws 5510 in the first rotary direction FRD to move the second frame 5200 from the retracted position (FIG. 16) toward the extended position (FIG. 17) in response to rotation of the drive gear 5410 by the Bowden cable 4010.

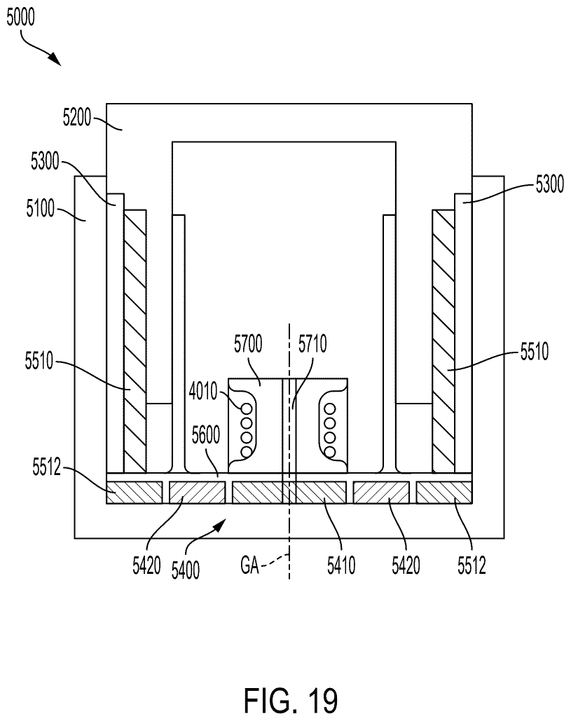

Referring to FIG. 19, in an alternative aspect, the pylon emulator 5000 includes a cover 5600 and a spindle (e.g., a wire spindle 5700). The cover 5600 covers the gear arrangement 5400. The wire spindle 5700 is operably engaged with the drive gear 5410 through the cover 5600. Specifically, a shaft 5710 extends through a hole in the cover 5600 and engages the drive gear 5410 such that the wire spindle 5700 and the drive gear 5410 rotate together. In one alternative aspect, the Bowden cable 4010 is wrapped around and affixed to the wire spindle 5700 in lieu of being attached directly to the drive gear 5410. In such an instance, when the Bowden cable 4010 is pulled in the first direction, the wire spindle 5700 and the drive gear 5410 will rotate about the drive gear axis GA. Further, in at least one aspect, the gear arrangement 5400 may further include drive screw gears 5512 operably engaged with the one or more than one drive screws 5510. The drive screw gears 5512 are covered by the cover 5600 and transmit their rotational motion through holes in the cover to their respective drive screws 5510. In use, actuation (e.g., pulling) of the Bowden cable 4010 in the first direction will rotate the wire spindle 5700 and the drive gear 5410 about the drive gear axis GA. The one or more than one intermediate gear 5420 will rotate in response to the rotation of the drive gear 5410, and the one or more than one drive screw 5510 will rotate in the first rotary direction FRD to move the second frame 5200 from the retracted position to the extended position in response to the rotation of the one or more than one intermediate gear 5420.

Referring to FIG. 14, in at least one aspect, a compression spring 5115 may be placed intermediate the first frame 5100 and the second frame 5200, similar to the compression spring 900 described with regard to FIG. 13. Specifically, the first frame 5100 can define an opening 5113 (FIG. 14) which may be utilized similar to the opening 113 described above. Further, an extension tab 5116 may extend from one of the walls 5212 of the second frame 5200 into the opening 5113 in the first frame 5100. The compression spring 5115 can be placed intermediate a top surface 5114 of the opening 5113 to bias the second frame 5200 toward the retracted position (e.g., toward the base plate 5110 of the first frame 5100). In instances where a user's extremity is operably attached to the second frame 200, the compression spring can aid the user in returning the second frame 200 to the retracted position.

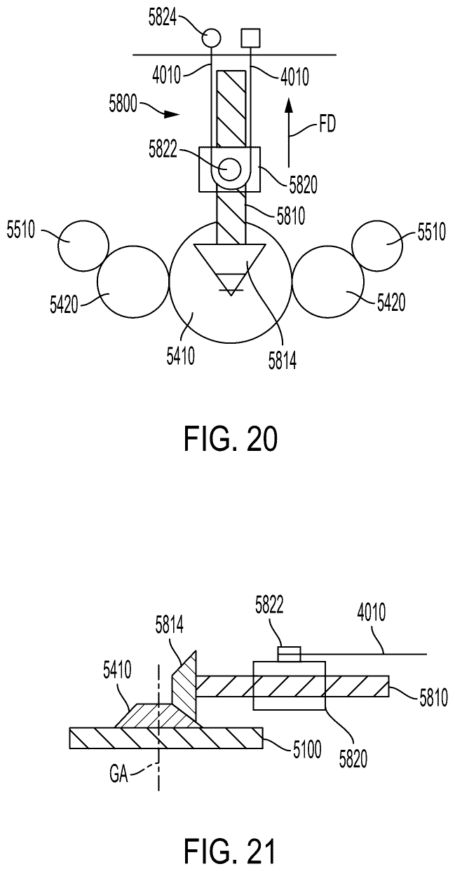

Referring to FIGS. 20 and 21, in one alternative aspect, the drive gear 5410 may be driven (e.g., rotated about the drive gear axis GA) by a second ball screw assembly 5800 that is oriented orthogonal to the one or more than one ball screw assembly 5500. The second ball screw assembly 5800 includes a second drive screw 5810 operably engaged with the one or more than one drive screw 5510 of the ball screw assembly 5500. The second ball screw assembly 5800 includes a housing 5820 that is slidable relative to the second drive screw 5810. In at least one aspect, a Bowden cable (e.g., the Bowden cable 4010) extends from the actuation and control system 4000 and is routed around a pin 5822 extending from the housing 5820. The cable 4010 terminates at a fixed position 5824 on the first frame 5100. As such, when the cable 4010 is pulled in the first direction FD, the housing 5820 will translate along the second drive screw 5810 away from the drive gear 5410. Further, the second ball screw assembly 5800 includes a plurality of ball bearings (not shown) positioned intermediate the second drive screw 5810 and the housing 5820. The ball bearings permit the second drive screw 5810 to rotate in response to translation of the housing 5820 along the second drive screw 5810. Further, in at least one aspect, the drive gear 5410 may be a bevel gear (see FIG. 21) and the second drive screw 5810 can include a bevel gear 5814 engaged with the bevel drive gear 5410. As such, rotation of the second drive screw 5810 will effectuate rotation of the drive gear 5410 about the drive gear axis GA.

In use, according to various embodiments, the housing 5820 is translated relative to the second drive screw 5810 in response to the Bowden cable 4010 being pulled in the first direction FD. The second drive screw 5810 is configured to rotate in response to the translation of the housing 5820. The drive gear 5410 is configured to rotate about the drive gear axis GA in response to rotation of the second drive screw 5810. Further, as discussed above, the one or more than one intermediate gear 5420, and the one or more than one drive screw 5510 are configured to rotate in response to the rotation of the drive gear 5410. As such, the first drive screw 5510 is configured to rotate in the first rotary direction FRD to move the second frame 5200 from the retracted position toward the extended position in response to rotation of the second drive screw 5810.

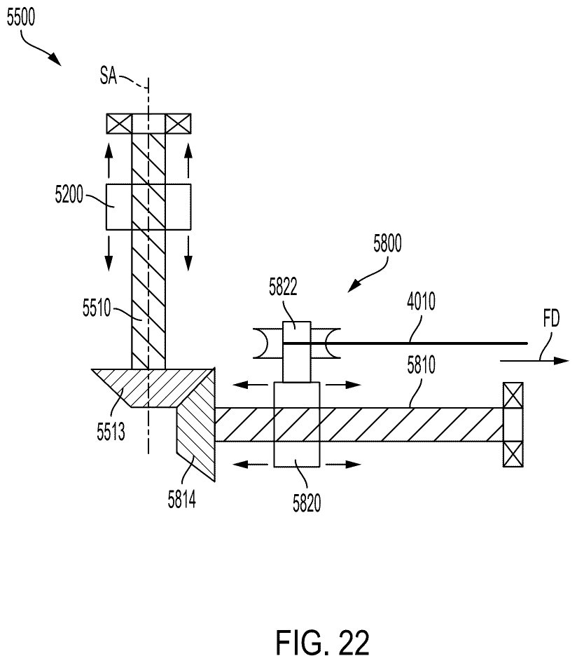

Further to the above, in one alternative aspect, one of the one or more than one drive screws 5510 may be directly engaged with the second ball screw assembly 5800 to move the second frame 200 from the retracted position to the extended position. Specifically, referring to FIG. 22, the drive screw 5510 can include a bevel gear 5513 engaged with the bevel gear 5814 of the second drive screw 5810 of the second ball screw assembly 5800. As such, when the Bowden cable 4010 is translated in the first direction FD by the actuation and control system 4000, for example, the housing 5820 will translate in the first direction FD. The second drive screw 5810 will rotate in response to the translation of the housing 5820 in the first direction, and the drive screw 5510 will rotate in the first rotary direction FRD to move the second frame 5200 from the retracted position to the extended position.

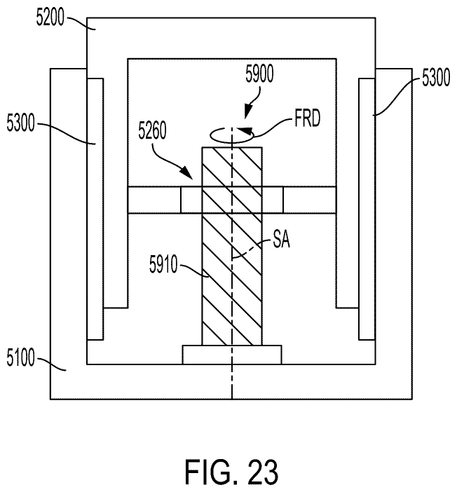

Further to the above, in one alternative aspect, the pylon emulator 5000 can include a central ball screw assembly as an alternative, or in addition to, the ball screw assemblies 5500, 5800 described above. Referring to FIG. 23, in at least one aspect, the pylon emulator 5000 can include a central ball screw assembly 5900 comprising a central drive screw 5910. The central drive screw 5910 is received in a central opening 5260 defined in the second frame 5200. Ball bearings (not shown) are positioned between the central opening 5260 and the drive screw 5910 to permit the second frame 5200 to translate along the central drive screw 5910 upon rotation of the central drive screw 5910 in the first rotary direction FRD about its screw axis SA. The central drive screw 5910 may be rotated about the screw axis SA by any suitable means, including those described herein. For example, the central drive screw 5910 may be rotated by attaching a Bowden cable directly to the central drive screw 5910. In such in instance, when the Bowden cable is pulled, the central drive screw 5910 will rotate in the first rotary direction FRD. In another alternative aspect, a drive gear, such as the drive gear 5410 described above, may be utilized to rotate the central drive screw 5910. Specifically, a Bowden cable may rotate the drive gear 5410 which, in turn, will rotate the central drive screw 5910 engaged with the drive gear 5410 as described in various aspects herein. Further, in another alternative aspect, a second ball screw assembly (e.g., the second ball screw assembly 5800) may be utilized to rotate the central drive screw 5910 as describe above in connection with FIG. 22.

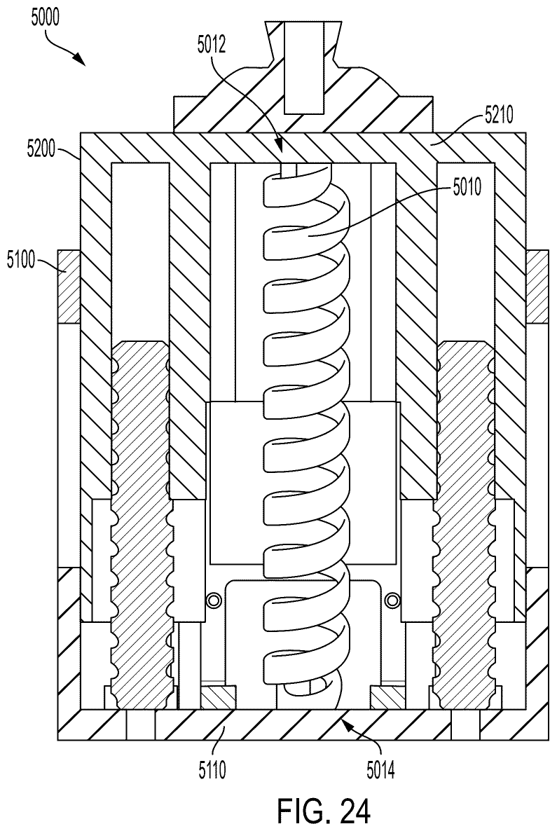

Referring to FIG. 24, in at least one aspect, the pylon emulator 5000 can include a biasing member (e.g., a coil spring 5010) extending between the second frame 5200 and the first frame 5100. In at least one aspect, a first end 5012 of the coil spring 5010 is attached to the top plate 5210 of the second frame 5200 and a second end 5014 of the coil spring 5010 is attached to the base plate 5110 of the first frame 5100. In an alternative aspect, the second end 5014 of the coil spring 5010 may be attached to the cover 5600 described above. In use, the coil spring 5010 biases the second frame 5200 toward the extended position. In various aspects, the coil spring 5010 biasing force is applied to the second frame 5200 in addition to the force applied to the second frame 5200 by the driver (e.g., the one or more than one ball screw assembly 5500) to move the second frame 5200 from the retracted position to the extended position. In various aspects, the coil spring 5010 provides some amount of stiffness to the pylon emulator 5000 and may aid the user in moving the second frame 200 to the extended position. Specifically, in instances where a user's extremity (e.g., the users leg) is operably attached to the second frame 5200, the coil spring 5010 can provide a biasing force to the users extremity to aid in moving the second frame 200 to the extended position. The coil spring 5010 arrangement described herein may be used with any of the pylon emulators described herein.

Referring again to FIGS. 16 and 17, in at least one aspect, the pylon emulator 5000 includes one or more than one load cell 5005 positioned intermediate the one or more than one drive screw 5510 and the second frame 5200. The load cell 5005 is configured to measure an amount of force applied by the drive screw 5510 to the second frame 5200 when the second frame 5200 moves from the retracted position toward the extended position in response to rotation of the drive screw 5510 in the first rotary direction FRD. In at least one aspect, the load cell 5005 is defined into the second frame 5200 such that the drive screw 5510 will abut a pressure plate of the load cell 5005. As the drive screw 5510 rotates, the amount of force applied by the drive screw 5510 to the second frame 5200 will be measured by the load cell 5005.

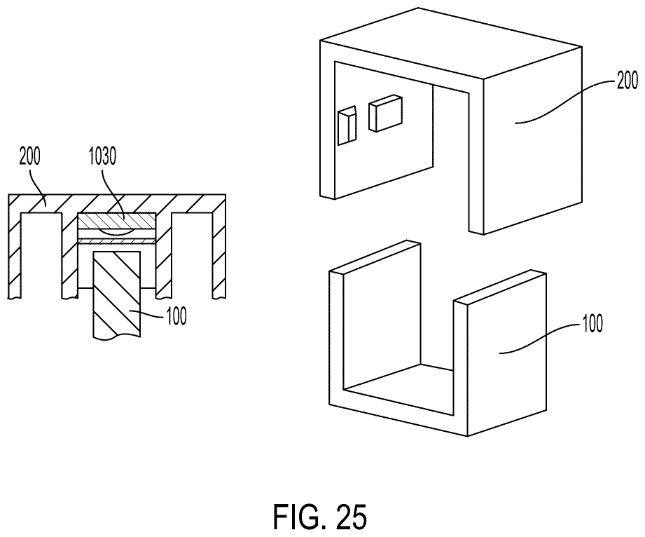

Referring to FIG. 25, in at least one aspect, the pylon emulator 1000 and/or the pylon emulator 5000 can include a compression load cell 1030. In at least one aspect, the compression load cell 1030 may be used in lieu of, or in addition to, the load cell 400 described above. In various aspects, the compression load cell 1030 may be positioned intermediate the first frame 100 and the second frame 200 as shown in FIG. 25. The compression load cell 1030 measures the force the first frame 100 applies to the second frame 200 as the second frame 200 moves between the retracted position and the extended position. In such instances, the first frame 100 and the second frame 200 may be separable.

In at least one embodiment, one or more compression load cells, such as the compression load cell 1030, may be positioned underneath one or more of the drive screws disclosed herein (e.g., such as the drive screw 5510) of the pylon emulator 5000. See FIG. 16. In at least one embodiment, a compression load cell can be positioned intermediate the drive screw 5510 and the base 5110 of the first frame 5100. Additionally, another compression load cell can be positioned between the second drive screw 5510 and the base 5110 of the first frame 5100.

In at least one embodiment, one or more compression load cells, such as the compression load cell 1030, may be positioned underneath a pyramid adapter (e.g., such as the pyramid adapter 1020) of the pylon emulator 1000 and/or the pylon emulator 5000. In at least one aspect, the compression load cell is positioned intermediate the top plate 5210 of the second frame 5200 and the pyramid adapter 1020 such that only a vertical load is applied to the compression load cell. In at least one aspect, the pyramid adapter 1020 is affixed to an addition plate that is positioned above the top plate 5210, and the additional plate is slidably attached to the top plate 5210 such that the additional plate and the pyramid adapter 1020 can float vertically relative to the top plate 5210. In such an instance, the compression load cell may be positioned intermediate the top plate 5210 and the additional plate such that only a vertical load is applied to the compression load cell during actuation of the pylon emulator 5000.

In at least one embodiment, the pyramid adapter 1020 is an instrumented pyramid adapter used to measure load for the pylon emulator 1000 and/or the pylon emulator 5000. In such instances, the instrumented pyramid adapter can include a load cell to measure the force applied to the instrumented pyramid adapter.

In various aspects, the pylon emulators described herein are able to exhibit software-controllable force-deflection characteristics using onboard sensors and off board actuation and control with a range and/or precision of adjustability far beyond conventional prosthetic pylon technology. Such programmable force-deflection behavior includes a linear or non-linear spring force profile, time based force profile, and/or other suitable force profiles. Onboard sensors include, for example, a load sensor, a strain gauge, a load cell with a position sensor, a string potentiometer, and/or any suitable combination thereof. Off board actuation allows for large loads to be applied while also allowing for the device to be of a minimal weight.

The pylon emulators described herein provide one degree-of-freedom (DoF) linear motion along a length of a user's leg. Stated another way, the pylon emulators model an axial DoF along the pylon of a transtibial amputee, for example, to control the instantaneous longitudinal stiffness, force, and/or displacement. The pylon emulators may include a string potentiometer to constantly monitor a displacement of the pylon emulator. The string potentiometer directly measures the displacement. Such a string potentiometer is able to withstand rapid accelerations and keep up with the rapid motions of the pylon emulator.

The above described design of the pylon emulator 1000 was tested and validated by implanting a linear stiffness controller that reacted to the measured displacement of the emulator 1000 and responded with a desired force. A velocity controller was used based on the difference between measured and desired force multiplied by a constant gain to send a velocity command to the actuator (e.g., the motor 4020). As described above, when the actuator is rotated, it pulls on a rope, or Bowden cable, which extends the emulator, or resists the compression of the emulator. In at least one aspect, a spring is used to maintain tension on the cable from the actuator.

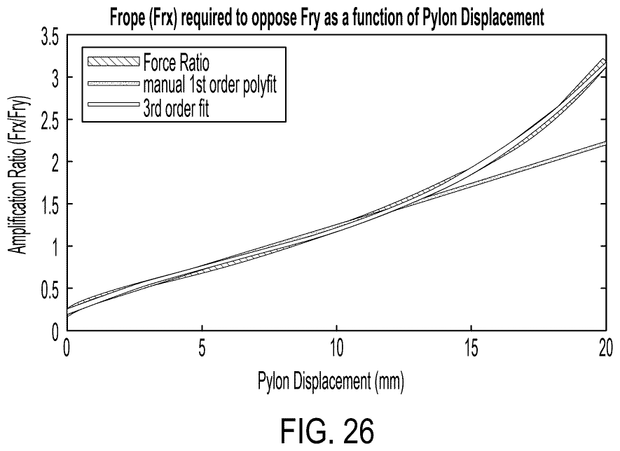

During testing and validation, the relation of force along an axial length of the pylon versus required force from the rope, or Bowden cable, due to the linkage was calculated to identify if control issues could exist. Given the disclosed geometry, there is a region of compression beyond which the required rope force increases exponentially resulting in a difficult region of control. As shown in FIG. 26, prior to reaching that region, the relationship between pylon displacement and required rope force is substantially linear. Such a difficult region of control could be counteracted by using a connection different from the linkage, such as a rack and pinion gear or a leadscrew, for example.

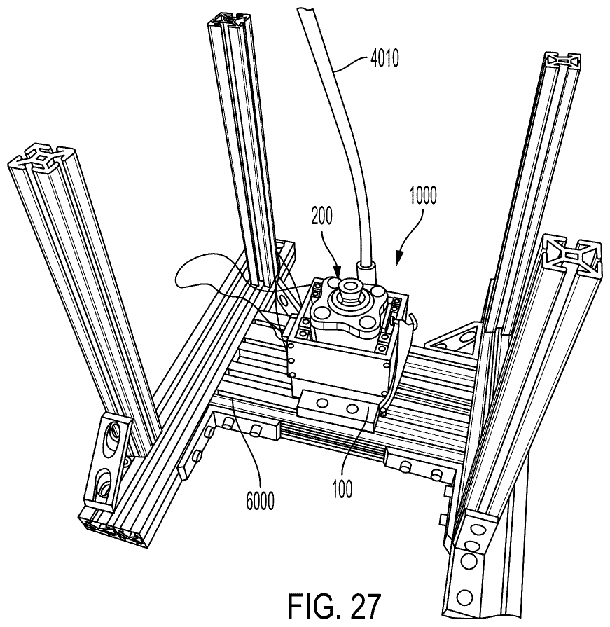

Testing and validation was performed for the pylon emulator 1000 using a rig 6000 as shown in FIG. 27. The rig 6000 is configured to hold the first frame 100 of the pylon emulator 1000 in position as a pre-determined, or otherwise known, load was applied to the second frame 200 of the pylon emulator acting to compress the emulator (e.g., acting to move the second frame 200 toward the retracted position). The rig 6000 comprises a rigid metal structure and an adapter plate to couple to the pylon emulator 1000 in substantially the same manner as the pylon emulator 1000 would couple to an ankle prosthesis, for example. The rig 6000 can further serve to track force control bandwidth by constraining the pylon emulator 1000 to fixed compression ranges.

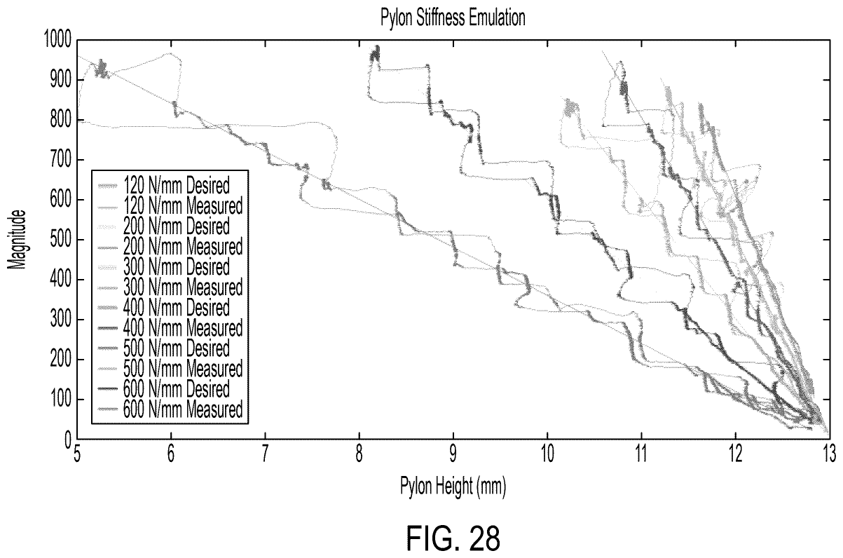

The pylon emulator 1000 is able to produce fine-tune adjustments in axial stiffness and/or other stiffness characteristics. During testing, a range of desired linear stiffnesses were applied in the controller and weight was slowly applied by hand to the pylon emulator 1000. As depicted in the graph of FIG. 28, entitled “Pylon Stiffness Emulation”, the pylon emulator 1000 was used to simulate a range of linear stiffness based on existing commercial pylons. The graph represents the magnitude of the stiffness measured in N versus the height of the pylon (e.g., the position of the second frame 200 relative to the first frame 100) in millimeters. The range of linear stiffness spans from 120 N/mm to 600 N/mm. As depicted, the pylon emulator 1000 exhibited accurate force tracking in the stiffest profiles with force tracking accuracy deviating slightly as the stiffness decreased. Such a drop-off in accuracy can be curtailed by improvements to the controller.

Example 1—In one various aspect, therefore, the present invention is directed to a prosthetic pylon emulator for use with a user and an off-board actuation and control system, the user having a pylon socket, the prosthetic pylon emulator comprising a first frame, a second frame, a load cell, a driver, and a Bowden cable. The first frame is operably attachable to a prosthesis, the first frame comprising a base plate and a pair of upstanding sidewalls. The second frame is operably attachable to the pylon socket of the user, the second frame is slidable relative to the first frame. The first frame is operably coupled to an upper portion of the load cell and the second frame is operably coupled to a lower portion of the load cell. The driver is operably engaged with the first frame and the second frame, the driver is configured to move the second frame from a retracted position to an extended position relative to the first frame upon actuation of the driver. The second frame is positioned farther away from the first frame when the second frame is in the extended position than when the second frame is in the retracted position. The Bowden cable is operably engaged with the driver and the off-board actuation and control system. The off-board actuation and control system is configured to pull the Bowden cable in a first direction to actuate the driver and move the second frame from the retracted position to the extended position.

Example 2—The prosthetic pylon emulator of Example 1, further comprising a biasing member, a first portion of the biasing member is removably attached to the first frame and a second portion of the biasing member is removably attached to the second frame such that the biasing member biases the second frame toward the retracted position.

Example 3—The prosthetic pylon emulator of Example 2, wherein the biasing member is an elastic band.

Example 4—The prosthetic pylon emulator of Example 1, 2, or 3, the second frame comprising an extension tab extending into an opening defined in the first frame, the pylon emulator further comprising a compression spring positioned intermediate the extension tab and an upper surface of the opening, the compression spring biases the second frame toward the retracted position.

Example 5—The prosthetic pylon emulator of Example 1, 2, 3, or 4, further comprising a biasing member, a first end of the biasing member is attached to a top plate of the second frame and a second end of the biasing member is attached to the base plate of the first frame, wherein the biasing member biases the second frame toward the extended position.

Example 6—The prosthetic pylon emulator of Example 5, wherein the biasing member is a coil spring.

Example 7—The prosthetic pylon emulator of Examples 1, 2, 3, 4, 5, or 6, wherein the second frame moves a stroke distance from the retracted position toward the extended position in response to the off board actuation and control system pulling the Bowden cable a first amount in the first direction, the pylon emulator further comprises a string potentiometer to determine the stroke distance based upon the off board actuation and control system pulling the Bowden cable the first amount.

Example 8—The prosthetic pylon emulator of Example 7, wherein a first portion of the string potentiometer being attached to the first frame and a second portion of the string potentiometer is attached to the second frame.

Example 9—The prosthetic pylon emulator of Examples 1, 2, 3, 4, 5, 6, 7, or 8, wherein the driver comprises a carriage assembly operably attached to the first frame and the second frame, the carriage assembly comprises a base carriage, an upper carriage, and one or more than one arm. The base carriage is slidably attached to the base plate of the first frame, the base carriage is linearly translatable relative to the base plate between a distal position and a proximal position. The Bowden cable is fixedly attached to the base carriage. The upper carriage is slidably attached to the second frame. The one or more than one arm is rotatably attached to the base carriage and rotatably attached to the upper carriage, the base carriage is moved from the distal position to the proximal position in response to the off-board actuation and control system pulling the Bowden cable in the first direction, and the one or more than one arm transitions from a collapsed configuration to an erect configuration to raise the upper carriage and move the second frame from the retracted position to the extended position in response to the base carriage moving from the distal position to the proximal position.

Example 10—The prosthetic pylon emulator of Example 9, wherein the upper carriage comprising an upper plate, a lower plate, and a plurality of bearing slides connecting the upper plate to the lower plate. The plurality of bearing slides are positioned intermediate the upper carriage and the second frame to permit the upper carriage to linearly translate relative to the second frame.

Example 11—The prosthetic pylon emulator of Example 10, wherein the second frame comprising a retention plate positioned intermediate the upper plate and the lower plate of the upper carriage. The upper portion of the load cell is retained to the upper plate and the lower portion of the load cell is retained to the retention plate.

Example 12—The prosthetic pylon emulator of Examples 9, 10, or 11, wherein the load cell is to measure an amount of force applied by the upper carriage to the second frame in response to the off board actuation and control system pulling the Bowden cable a first amount in the first direction.

Example 13—The prosthetic pylon emulator of Examples 9, 10, 11, or 12, wherein the load cell is placed in tension in response to the off board actuation and control system pulling the Bowden cable the first amount in the first direction.

Example 14—The prosthetic pylon emulator of Examples 1, 2, 3, 4, 5, 6, 7, or 8, wherein the driver comprising a ball screw assembly operably engaged with the Bowden cable, the ball screw assembly comprising a drive screw and a plurality of ball bearings. The drive screw is rotatably attached to the first frame and operably engaged with the Bowden cable. The drive screw extends into an internal housing defined in the second frame. The plurality of ball bearings are positioned intermediate the drive screw and the internal housing of the second frame. The drive screw is to rotate in a first rotary direction in response to the off-board actuation and control system pulling the Bowden cable in the first direction. Rotation of the drive screw in the first rotary direction moves the second frame from the retracted position toward the extended position.

Example 15—The prosthetic pylon emulator of Example 14, further comprising a gear arrangement comprising a drive gear and an intermediate gear. The drive gear is rotatably attached to the first frame. The Bowden cable is configured to rotate the drive gear in response to the off-board actuation and control system pulling the Bowden cable in the first direction. The intermediate gear is rotatably attached to the first frame and engaged with the drive gear and the drive screw. The intermediate gear rotates the drive screw in the first rotary direction to move the second frame from the retracted position to the extended position in response to rotation of the drive gear.

Example 16—The prosthetic pylon emulator of Example 15, further comprising a second ball screw assembly operably engaged with the drive gear, wherein a second drive screw of the second ball screw assembly is configured to rotate in response to the off board actuation and control system pulling the Bowden cable in the first direction, and wherein the drive gear is configured to rotate in response to the rotation of the second drive screw.

Example 17—The prosthetic pylon emulator of Examples 14, 15, or 16, comprising a load cell positioned intermediate the one or more than one drive screw and the internal housing of the second frame, the load cell is configured to measure an amount of force applied by the drive screw to the second frame when the second frame moves from the retracted position toward the extended position.

Example 18—The prosthetic pylon emulator of Example 1, 2, 3, 4, 5, 6, 7, or 8, wherein the driver comprises a first ball screw assembly and a second ball screw assembly. The first ball screw assembly comprising a first drive screw rotatably attached to the first frame, the first drive screw extending into an internal housing defined in the second frame, and a plurality of ball bearings positioned intermediate the first drive screw and the internal housing of the second frame. The second ball screw assembly comprises a second drive screw operably engaged with the first drive screw, the second drive screw positioned orthogonal to the first drive screw, a housing slidable relative to the second drive screw, the Bowden cable is attached to the housing, and a plurality of ball bearings are positioned intermediate the second drive screw and the housing. The housing is translated relative to the second drive screw in response to the off-board actuation and control system pulling the Bowden cable in the first direction. The second drive screw is configured to rotate in response to the translation of the housing, the first drive screw is configured to rotate in the a first rotary direction to move the second frame from the retracted position toward the extended position in response to rotation of the second drive screw.

Example 19—The prosthetic pylon emulator of Example 18, wherein the first drive screw and the second drive screw are operably engaged by a bevel gear arrangement.

Example 20—The prosthetic pylon emulator of Example 18 or 19, further comprising a load cell positioned intermediate the first drive screw and the second frame, the load cell is configured to measure an amount of force applied by the first drive screw to the second frame when the second frame moves from the retracted position toward the extended position.

The examples presented herein are intended to illustrate potential and specific implementations of the present invention. It can be appreciated that the examples are intended primarily for purposes of illustration of the invention for those skilled in the art. No particular aspect or aspects of the examples are necessarily intended to limit the scope of the present invention. Further, it is to be understood that the figures and descriptions of the present invention have been simplified to illustrate elements that are relevant for a clear understanding of the present invention, while eliminating, for purposes of clarity, other elements. While various embodiments have been described herein, it should be apparent that various modifications, alterations, and adaptations to those embodiments may occur to persons skilled in the art with attainment of at least some of the advantages. Persons skilled in the art will appreciate that recited operations therein may generally be performed in any order. Also, although various operational flow diagrams are presented in a sequence(s), it should be understood that the various operations may be performed in other orders than those which are illustrated, or may be performed concurrently. The disclosed embodiments are therefore intended to include all such modifications, alterations, and adaptations without departing from the scope of the embodiments as set forth herein.

What is claimed is:

1. A prosthetic pylon emulator for use with a user and an off-board actuation and control system, the user having a pylon socket, the prosthetic pylon emulator comprising:
a first frame operably attachable to a prosthesis, the first frame comprising a base plate and a pair of upstanding sidewalls;
a second frame operably attachable to the pylon socket of the user, wherein the second frame is slidable relative to the first frame;
a load cell, wherein the first frame is operably coupled to an upper portion of the load cell and the second frame is operably coupled to a lower portion of the load cell;
a driver operably engaged with the first frame and the second frame, the driver configured to move the second frame from a retracted position to an extended position relative to the first frame upon actuation of the driver, wherein the second frame is positioned farther away from the first frame when the second frame is in the extended position than when the second frame is in the retracted position; and
a Bowden cable operably engaged with the driver and the off-board actuation and control system, wherein the off-board actuation and control system is configured to pull the Bowden cable in a first direction to actuate the driver and move the second frame from the retracted position to the extended position.

2. The prosthetic pylon emulator of claim 1, further comprising a biasing member, a first portion of the biasing member is removably attached to the first frame and a second portion of the biasing member is removably attached to the second frame such that the biasing member biases the second frame toward the retracted position.

3. The prosthetic pylon emulator of claim 2, wherein the biasing member is an elastic band.

4. The prosthetic pylon emulator of claim 1, wherein the second frame comprises an extension tab extending into an opening defined in the first frame, the pylon emulator further comprising a compression spring positioned intermediate the extension tab and an upper surface of the opening, the compression spring biases the second frame toward the retracted position.

5. The prosthetic pylon emulator of claim 1, further comprising a biasing member, a first end of the biasing member is attached to a top plate of the second frame and a second end of the biasing member is attached to the base plate of the first frame, wherein the biasing member biases the second frame toward the extended position.

6. The prosthetic pylon emulator of claim 5, wherein the biasing member is a coil spring.

7. The prosthetic pylon emulator of claim 1, wherein the second frame moves a stroke distance from the retracted position toward the extended position in response to the off board actuation and control system pulling the Bowden cable a first amount in the first direction, the pylon emulator further comprises a string potentiometer to determine the stroke distance based upon the off board actuation and control system pulling the Bowden cable the first amount.

8. The prosthetic pylon emulator of claim 7, wherein a first portion of the string potentiometer is attached to the first frame and a second portion of the string potentiometer is attached to the second frame.

9. The prosthetic pylon emulator of claim 1, wherein the driver comprises a carriage assembly operably attached to the first frame and the second frame, the carriage assembly comprises:
a base carriage slidably attached to the base plate of the first frame, the base carriage is linearly translatable relative to the base plate between a distal position and a proximal position, wherein the Bowden cable is fixedly attached to the base carriage;
an upper carriage slidably attached to the second frame; and
one or more than one arm rotatably attached to the base carriage and rotatably attached to the upper carriage, wherein the base carriage is moved from the distal position to the proximal position in response to the off-board actuation and control system pulling the Bowden cable in the first direction, and wherein the one or more than one arm transitions from a collapsed configuration to an erect configuration to raise the upper carriage and move the second frame from the retracted position to the extended position in response to the base carriage moving from the distal position to the proximal position.

10. The prosthetic pylon emulator of claim 9, wherein the upper carriage comprises:
an upper plate;
a lower plate; and
a plurality of bearing slides connecting the upper plate to the lower plate, the plurality of bearing slides positioned intermediate the upper carriage and the second frame to permit the upper carriage to linearly translate relative to the second frame.

11. The prosthetic pylon emulator of claim 10, wherein the second frame comprise a retention plate positioned intermediate the upper plate and the lower plate of the upper carriage, wherein the upper portion of the load cell is retained to the upper plate and the lower portion of the load cell is retained to the retention plate.

12. The prosthetic pylon emulator of claim 11, wherein the load cell is to measure an amount of force applied by the upper carriage to the second frame in response to the off board actuation and control system pulling the Bowden cable a first amount in the first direction.

13. The prosthetic pylon emulator of claim 12, wherein the load cell is placed in tension in response to the off board actuation and control system pulling the Bowden cable the first amount in the first direction.

14. The prosthetic pylon emulator of claim 1, wherein the driver comprises a ball screw assembly operably engaged with the Bowden cable, the ball screw assembly comprising:
a drive screw rotatably attached to the first frame and operably engaged with the Bowden cable, the drive screw extends into an internal housing defined in the second frame; and
a plurality of ball bearings positioned intermediate the drive screw and the internal housing of the second frame, wherein the drive screw is to rotate in a first rotary direction in response to the off-board actuation and control system pulling the Bowden cable in the first direction, and wherein rotation of the drive screw in the first rotary direction moves the second frame from the retracted position toward the extended position.

15. The prosthetic pylon emulator of claim 14, further comprising a gear arrangement, comprising:
a drive gear rotatably attached to the first frame, wherein the Bowden cable is configured to rotate the drive gear in response to the off-board actuation and control system pulling the Bowden cable in the first direction; and
an intermediate gear rotatably attached to the first frame and engaged with the drive gear and the drive screw, wherein the intermediate gear rotates the drive screw in the first rotary direction to move the second frame from the retracted position to the extended position in response to rotation of the drive gear.

16. The prosthetic pylon emulator of claim 15, further comprising a second ball screw assembly operably engaged with the drive gear, wherein a second drive screw of the second ball screw assembly is configured to rotate in response to the off board actuation and control system pulling the Bowden cable in the first direction, and wherein the drive gear is configured to rotate in response to the rotation of the second drive screw.

17. The prosthetic pylon emulator of claim 14, further comprising a load cell positioned intermediate the one or more than one drive screw and the internal housing of the second frame, the load cell is configured to measure an amount of force applied by the drive screw to the second frame when the second frame moves from the retracted position toward the extended position.

18. The prosthetic pylon emulator of claim 1, wherein the driver comprises:
a first ball screw assembly, comprising:
a first drive screw rotatably attached to the first frame, the first drive screw extending into an internal housing defined in the second frame; and
a plurality of ball bearings positioned intermediate the first drive screw and the internal housing of the second frame; and
a second ball screw assembly, comprising:
a second drive screw operably engaged with the first drive screw, the second drive screw positioned orthogonal to the first drive screw;
a housing slidable relative to the second drive screw, the Bowden cable attached to the housing; and
a plurality of ball bearings positioned intermediate the second drive screw and the housing, wherein the housing is translated relative to the second drive screw in response to the off-board actuation and control system pulling the Bowden cable in the first direction, wherein the second drive screw is configured to rotate in response to the translation of the housing, and wherein the first drive screw is configured to rotate in the a first rotary direction to move the second frame from the retracted position toward the extended position in response to rotation of the second drive screw.

19. The prosthetic pylon emulator of claim 18, wherein the first drive screw and the second drive screw are operably engaged by a bevel gear arrangement.

20. The prosthetic pylon emulator of claim 18, further comprising a load cell positioned intermediate the first drive screw and the second frame, the load cell is configured to measure an amount of force applied by the first drive screw to the second frame when the second frame moves from the retracted position toward the extended position.
